(19) United States
(12) Patent Application Publication
(10) Pub. No.: US 2025/0364079 A1
(43) Pub. Date: Nov. 27, 2025

(54) PREDICTING LABELS FOR BIOLOGICAL SEQUENCES USING NEURAL NETWORKS CONDITIONED ON POSITIVE AND NEGATIVE EXAMPLES

(71) Applicant: DeepMind Technologies Limited, London (GB)

(72) Inventors: Peter Thomas Shaw, Seattle, WA (US); Bhaskar Srinivas Gurram, Mountain View, CA (US); David Benjamin Belanger, Cambridge, MA (US); Georgiana Andreea Gane, Mountain View, CA (US); Maxwell Bileschi, Waltham, MA (US); Lucy Colwell, Cambridge, MA (US); Kristina Nikolova Toutanova, Redmond, WA (US); Ankur P. Parikh, New York, NY (US)

(21) Appl. No.: 19/209,989

(22) Filed: May 16, 2025

Related U.S. Application Data
(60) Provisional application No. 63/650,715, filed on May 22, 2024.

(51) Int. Cl. G16B 30/10; G16B 15/30; G16B 40/20; G16B 45/00

(52) U.S. Cl. CPC G16B 30/10; G16B 15/30; G16B 40/20; G16B 45/00

(57) ABSTRACT

Methods, systems, and apparatus, including computer programs encoded on a computer storage medium, for predicting labels for biological sequences. One of the methods includes, in response to receiving a request to identify labels associated with an input biological sequence: determining, for each of a plurality of candidate labels, a score characterizing a likelihood that the input biological sequence is associated with the candidate label. Each score is determined by identifying a plurality of positive biological sequences that are each associated with the candidate label; and processing a network input including the input biological sequence and the plurality of positive biological sequences using a neural network to generate the score characterizing the likelihood that the input biological sequence is associated with the candidate label. The method includes selecting one or more of the candidate labels as labels for the input biological sequence based on the scores.

### CROSS-REFERENCE TO RELATED APPLICATIONS

This application claims priority from U.S. Provisional Application No. 63/650,715, filed on May 22, 2024, the entire contents of which are incorporated by reference herein.

### BACKGROUND

This specification relates to processing data using machine learning models.

Machine learning models receive an input and generate an output, e.g., a predicted output, based on the received input. Some machine learning models are parametric models and generate the output based on the received input and on values of the parameters of the model.

Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification generally describes a system implemented as computer programs on one or more computers in one or more locations that can identify one or more labels for an input biological sequence.

According to one aspect, there is provided a method performed by one or more computers, the method comprising: receiving a request to identify one or more labels associated with an input biological sequence; and in response to receiving the request: determining, for each of a plurality of candidate labels, a score characterizing a likelihood that the input biological sequence is associated with the candidate label, comprising, for each of the plurality of candidate labels: identifying a plurality of positive biological sequences that are each associated with the candidate label; and processing a network input comprising: (i) the input biological sequence, and (ii) the plurality of positive biological sequences, using a neural network and in accordance with values of a set of neural network parameters to generate the score characterizing the likelihood that the input biological sequence is associated with the candidate label; and selecting one or more of plurality of candidate labels as labels for the input biological sequence based on the scores.

In some implementations, the method further comprises for each of the plurality of candidate labels, identifying a plurality of negative biological sequences that are each not associated with the candidate label; wherein for each of the plurality of candidate labels, the network input to the neural network further comprises the plurality of negative biological sequences.

In some implementations, for each of the plurality of candidate labels, the network input to the neural network includes labeling data that identifies each of the plurality of positive biological sequences as being associated with the candidate label. In some implementations, for each of the plurality of candidate labels, the network input to the neural network further comprises data identifying the candidate label.

In some implementations, for each of the plurality of candidate labels, the network input to the neural network further comprises one or more of: data characterizing a three-dimensional (3D) structure of a molecule includes the input biological sequence; or for one or more of the plurality of positive biological sequences, data characterizing a respective 3D structure of a molecule that includes the positive biological sequence. In some examples, selecting the plurality of positive biological sequences as a proper subset of the set of candidate positive biological sequences based on the similarity scores comprises: selecting a plurality of highest ranked candidate positive biological sequences under a ranking of the set of candidate positive biological sequences based on the similarity scores. In some examples, selecting the plurality of positive biological sequences as a proper subset of the set of candidate positive biological sequences based on the similarity scores comprises: stochastically sampling the plurality of positive biological sequences from the set of candidate positive biological sequences based on the similarity scores.

In some implementations, for each of the plurality of candidate labels, identifying the plurality of negative biological sequences that are each not associated with the candidate label comprises: determining, for each candidate negative biological sequence in a set of candidate negative biological sequences that are not associated with the candidate label, a respective similarity score that measures a similarity between: (i) the candidate negative biological sequence, and (ii) the input biological sequence; and selecting the plurality of negative biological sequences as a proper subset of the set of candidate negative biological sequences based on the similarity scores.

In some of these implementations, selecting the plurality of negative biological sequences as a proper subset of the set of candidate negative biological sequences based on the similarity scores comprises: selecting a plurality of highest ranked candidate negative biological sequences under a ranking of the set of candidate negative biological sequences based on the similarity scores. In some of these implementations, selecting the plurality of negative biological sequences as a proper subset of the set of candidate negative biological sequences based on the similarity scores comprises: stochastically sampling the plurality of negative biological sequences from the set of candidate negative biological sequences based on the similarity scores.

In some implementations, the neural network has been trained by operations comprising: pre-training the neural network to perform a pre-training task comprising processing a network input that includes a pair of training biological sequences to generate a predicted similarity score that is a prediction for a similarity between the pair of input biological sequences; and fine-tuning the neural network to perform a fine-tuning task of predicting labels for training biological sequences. In some examples, pre-training the neural network to perform the pre-training task further comprises: partially masking one or both training biological sequences of the pair of training biological sequences prior to providing the pair of training biological sequences as a network input to the neural network; and generating, using the neural network, a prediction for an unmasked version of any masked portions of the pair of training biological sequences.

In some implementations, the method further comprises, in response to receiving the request, selecting the plurality of candidate labels for the input biological sequence as a proper subset of a set of possible labels for the input biological sequence. In some examples, selecting the plurality of candidate labels for the input biological sequence as the proper subset of the set of possible labels for the input biological sequence comprises: determining, for each example biological sequence in a set of example biological sequences, a respective similarity score that measures a similarity between: (i) the example biological sequence, and (ii) the input biological sequence; selecting a proper subset of the set of example biological sequences based on the similarity scores; and identifying each label that is associated with at least one of the example biological sequences in the selected proper subset of the set of example biological sequences as a candidate label for the input biological sequence. In some examples, selecting the proper subset of the set of example biological sequences based on the scores comprises: selecting a plurality of highest ranked example biological sequences under a ranking of the set of example biological sequences based on the similarity scores.

In some implementations, processing the network input using the neural network comprises processing a representation of the network input as a sequence of embeddings using the neural network. In some implementations, the neural network comprises a plurality of attention layers. In some implementations, the neural network comprises an encoder-decoder Transformer architecture. In some implementations, the input biological sequence comprises an amino acid sequence of a protein.

In some implementations, the input biological sequence comprises a nucleotide sequence of a deoxyribonucleic acid (DNA) molecule. In some implementations, the input biological sequence comprises a nucleotide sequence of a ribonucleic acid (RNA) molecule.

In some implementations, the plurality of candidate labels include one or more biological function labels that each specify a respective biological function; wherein a biological sequence is associated with a biological function label if molecules including the biological sequence have the biological function specified by the biological function label. In some implementations, the plurality of candidate labels include one or more subcellular localization labels that each specify a subcellular location; wherein a biological sequence is associated with a subcellular localization label if molecules including the biological sequence are active in the subcellular location specified by the subcellular localization label.

In some implementations, the plurality of candidate labels include one or more enzymatic activity labels that each specify a respective type of reaction; wherein a biological sequence is associated with an enzymatic activity label if the molecules including the biological sequence are involved in catalyzing the type of reaction specified by the enzymatic activity label. In some implementations, the plurality of candidate labels include a solubility label; wherein a biological sequence is associated with the solubility label if molecules including the biological sequence are soluble.

In some implementations, the method (e.g. as described in the “one aspect” above) comprises: selecting the input biological sequence as (or otherwise defining) a drug target or substrate of an industrial enzyme based at least in part on the one or more labels selected for the input biological sequence; and identifying one or more ligands that are predicted to bind to a molecule that includes the input biological sequence, or which is otherwise defined by the input sequence. Thus, the method may be used to identify ligands that can act as drugs to provide a therapeutic effect, or as enzymes in an industrial process.

In some implementations, identifying one or more ligands that are predicted to bind to a molecule that includes the input biological sequence comprises, for each candidate ligand in a collection of candidate ligands: determining a predicted binding affinity of the ligand for the molecule that includes the input biological sequence; and determining whether to select the candidate ligand as a ligand that is predicted to bind to the molecule that includes the input biological sequence based at least in part on the predicted binding affinity.

In some implementations, (i) the molecule comprises a receptor, e.g. that includes the input biological sequence, and the identified one or more ligands that are predicted to bind to the molecule that includes the input biological sequence are agonists or antagonists of the receptor; or (ii) the molecule comprises an antibody or aptamer target that includes the input biological sequence, in particular a virus or cancer cell protein, and wherein the identified one or more ligands that are predicted to bind to the molecule are antibodies or aptamers that bind to the antibody or aptamer target to provide a therapeutic effect.

As particular examples, each ligand may be a polypeptide ligand, a polynucleoside ligand, or a polynucleotide ligand, or an antibody, or an aptamer.

In some implementations, the method, further comprises physically synthesizing the molecule that includes the input biological sequence for use in treating one or more diseases associated with a target molecule selected from the identified one or more candidate target molecules.

In some implementations, the method further comprises: performing physical experiments on physically synthesized instances of the drug molecule to determine one or more of: absorption properties of the ligand, or distribution properties of the ligand, or metabolism properties of the ligand, or excretion properties of the ligand, or toxicity properties of the ligand.

In some implementations, the method (e.g. of the “one aspect” above), further comprises: selecting the input biological sequence for inclusion in a molecule, or selecting a molecule that is otherwise defined by the input sequence, based at least in part on the one or more labels selected for the input biological sequence; and identifying one or more candidate target molecules to which a molecule that includes the input biological sequence is predicted to bind. The molecule that includes the input biological sequence may be a drug molecule or an industrial enzyme (or e.g. where the input biological sequence is a DNA or RNA sequence, a protein coded for by the DNA or RNA sequence) and each candidate target molecule may be a candidate target molecule of the drug molecule or a candidate substrate molecule of the industrial enzyme.

In some implementations, identifying one or more candidate target molecules to which the molecule that includes (or is defined/coded for by) the input biological sequence is predicted to bind comprises, for each candidate target molecule in a collection of candidate target molecules: determining a predicted binding affinity of the molecule that includes the input biological sequence for the candidate target molecule; and determining whether to select the candidate target molecule to which the molecule that includes (or is defined/coded for by) the input biological sequence is predicted to bind based at least in part on the predicted binding affinity.

In some implementations, (i) the molecule that includes (or is defined/coded for by) the input biological sequence is an agonist or antagonist of a receptor of the identified one or more target molecules; or (ii) the identified one or more target molecules each comprise a respective antibody or aptamer target, in particular a virus or cancer cell protein, and the molecule that includes the input biological sequence is an antibody or aptamer that binds to the antibody or aptamer target to provide a therapeutic effect. In some examples, the molecule that includes the input biological sequence is a polypeptide, a polynucleoside, or a polynucleotide, or an antibody, or an aptamer.

In some implementations, the method further comprises physically synthesizing the molecule that includes (or is defined/coded for by) the input biological sequence for use in treating one or more diseases associated with a target molecule selected from the identified one or more candidate target molecules.

In some examples, the molecule that includes (or is defined/coded for by) the input biological sequence is a drug molecule and the method further comprises, performing physical experiments on physically synthesized instances of the drug molecule to determine one or more of: absorption properties of the drug molecule, or distribution properties of the drug molecule, or metabolism properties of the drug molecule, or excretion properties of the drug molecule, or toxicity properties of the drug molecule.

In some implementations of the method (e.g., the method as described in the “one aspect” above), the method is for identifying the presence of one or more diseases and the input biological sequence is determined by analyzing a version of a protein or nucleic acid obtained from a human or animal body. The method may then comprise determining that the input biological sequence is associated with one or more diseases based at least in part on the one or more labels selected for the input biological sequence. As particular examples, the one or more diseases may comprise: a genetic disease; a protein mis-folding disease; or a nucleic acid mis-folding disease.

In some implementations, the input biological sequence includes an amino acid sequence of a protein, and the method further comprises: selecting the protein as a target for increasing or decreasing production of an output produced by a biochemical pathway based at least in part on the one or more labels selected for the input biological sequence of the protein; and determining one or more genetic edits to modulate expression of the protein. In some examples, the method further comprises applying the one or more genetic edits to a genome of an organism to modulate the expression of the protein.

Particular embodiments of the subject matter described in this specification can be implemented so as to realize one or more of the following advantages.

The system described in this specification can process an input biological sequence to determine a respective score for each of one or more candidate labels, and then select one or more of the candidate labels as labels for the input biological sequence based on the scores. The input biological sequence can be, e.g., an amino acid sequence of a protein molecule or a nucleotide sequence of a DNA or RNA molecule. The candidate labels for the biological sequence can characterize, e.g., a biological function of the biological sequence. Labels that the system identifies for an input biological sequence can be used, e.g., to identify a molecule that includes the input biological sequence as a drug target (e.g., a molecule, such as a protein, that is associated with one or more disease processes occurring within a living organism), or (in cases where the input biological sequence is an amino acid sequence of a protein) to identify the protein as a target for genetic edits to increase production of an output by a biochemical pathway that includes the protein.

One approach to identifying biological labels for an input biological sequence is a homology-based approach that involves determining a respective similarity of the input biological sequence to each example biological sequence in a large set of example biological sequences with known labels. The labels of the example biological sequences that are most similar to the input biological sequence can then be associated with the input biological sequence.

Another approach to identifying biological labels for an input biological sequence is a machine learning-based approach that involves generating biological labels as an output of a machine learning model that processes the input biological sequence and that has been trained on a set of labeled biological sequences using a supervised learning technique.

However, a critical challenge in labeling biological sequences is handling sequences and labels that are not well represented in the available training data. For instance, a substantial number of proteins belong to the “dark matter” of the protein universe, e.g., they are distant in sequence space from any characterized proteins. Both approaches described above for identifying biological labels may perform poorly when handling sequences and labels that are not well represented in the available training data. For instance, the homology-based approach may fail to identify any example biological sequences that have a high similarity to the input biological sequence, and propagating labels to the input biological sequence from example biological sequences with low similarity to the input biological sequence can result in inaccurate labeling. As another example, the machine learning-based approach may fail to generalize to out-of-distribution sequences and labels that are not well represented in the training data used for training the machine learning model.

The system described in this specification can address these issues. In particular, to determine the likelihood that an input biological sequence has a candidate label, the system can identify: (i) a set of one or more “positive” biological sequences that are associated with the candidate label, and (ii) a set of one or more “negative” biological sequences that are not associated with the candidate label. The system can then process a network input that includes the input biological sequence, the positive biological sequences, and the negative biological sequences using a neural network to generate a score defining a likelihood that the input biological sequence has the candidate label. The system can increase the relevance of the positive biological sequences and the negative biological sequences to the input biological sequence by selecting candidate positive/negative biological sequences based on their similarity to the input biological sequences. The neural network can implicitly identify and compare complex patterns and relationships among the input, positive, and negative biological sequences to accurately label the input biological sequence.

The system can address the challenges of the machine learning-based approach described above because the system uses a neural network that, instead of relying entirely on information encoded in the network parameters of the neural network, can explicitly leverage the positive and negative biological sequences included in the network input. Further, the system can address the challenges of the homology-based approach described above because, instead of propagating labels directly to the input biological sequence from positive biological sequences, the neural network performs machine learned operations that can implicitly account for potentially low similarity between the positive biological sequences and the input biological sequence.

The system can enable a reduction in consumption of computational resources (e.g., memory and computing power) compared to other approaches. For instance, one way to address the deficiencies of the alternative homology and machine learning-based approaches described above is to generate large ensembles of predictions using these approaches, e.g., using different random seeds, different sets of training data, different model architectures, and so forth. Such ensemble-based approaches can contribute to increasing accuracy but can also be hugely computationally intensive. In contrast, the system described in this specification can effectively generate labels for biological sequences without requiring an ensemble of separate and distinct instantiations of the system and can thus avoid the computational overhead associated with ensemble-based approaches.

In addition to training the neural network to perform the task of predicting labels for biological sequences, the system can additionally pre-train the neural network to perform the auxiliary task of predicting similarity between pairs of biological sequences, unmasking masked versions of pairs of biological sequences, or both in combination. Pre-training the neural network in this manner can enable the neural network to more efficiently learn relationships between an input biological sequence and positive/negative biological sequences, and in particular can increase the prediction accuracy of the neural network on the main task of labeling biological sequences. The pre-training can additionally increase the robustness and generalizability of the neural network and thus enable reduced consumption of computational resources (e.g., memory and computing power), e.g., by obviating the need for ensemble-based approaches, as described above.

### Screening

In general, implementations of the described techniques can be used to screen biological sequences for particular functions or associations. In general, the input biological sequence and/or the positive and negative biological sequences can comprise, e.g., an amino acid sequence, or a nucleic acid sequence such as RNA or DNA, or can be a sequence that codes for, e.g., represents a molecule (e.g. a protein that is synthesized in a living organism based on an RNA or DNA sequence).

Candidate input biological sequences can be screened for a particular biological function or property by choosing one of the candidates as the input biological sequence and identifying a plurality of positive (and/or negative) biological sequences that have (or lack) the particular biological function or property, and then processing the candidate as described herein. Since an amino acid or a nucleic acid sequence can define a molecule; e.g., in the examples described a molecule can be a molecule that is defined by a biological sequence; and a sequence can be one that defines (e.g., codes for) a particular molecule.

### Vaccines

Implementations of the method can be used to identify a vaccine. In general, a vaccine comprises a molecule or molecules with a particular shape, e.g., comprising a particular protein or having a particular surface feature from a virus or bacterium (and thus may comprise a weakened version of the virus or bacterium). Alternatively, a vaccine may comprise mRNA that codes for such a molecule.

The input biological sequence may comprise an amino acid sequence for a molecule such as a protein or part of such a molecule (e.g., so as to avoid inducing an undesirable response to a whole protein in an organism). Or the input biological sequence may comprise a nucleic acid sequence. The positive and/or negative biological sequences may similarly comprise an amino acid sequence or a nucleic acid sequence.

The positive biological sequences may be chosen so as to be sequences that give rise to an immune response. They can be chosen, e.g., based on a strength of the response and/or based on a function of an exposed part of a (folded) molecule defined by the sequence. As another example some of the positive biological sequences may be chosen as (short) parts of a sequence (molecule) that induces an immune response. For example, for a cell surface protein, 10% of the parts of sequence of the protein may be positive examples and 90% negative examples. The described techniques may then be used to identify other sequences or molecules that are also predicted to provoke an immune response.

### Motifs

More generally, implementations of the described techniques may be used to identify sequences or molecules that have a particular motif (e.g., related to a particular biological function), e.g., a structure motif or a structural motif encoded by the sequence. An example of such a motif is a zinc finger motif for a DNA-binding protein. The positive and negative examples can be chosen appropriately for the particular motif that is to be identified as associated with the input biological sequence.

### DNA Sequences

In some implementations the input biological sequence comprises a DNA sequence. The positive or negative sequences can also comprise DNA sequences, e.g., sequences with a similar function, e.g., from the same species or particular organism, or from different species. In the case of particular organism, the described techniques can be used for personalized medicine.

As an example, the DNA sequences may be regulatory sequences, e.g., repressors, or activators, or regulatory elements in general. Such regulatory sequences can regulate biological machinery in an organism, e.g., to turn the machinery on or off or, more generally, to regulate a degree of activity of the machinery. The machinery can, e.g., produce, or control production of, another molecule such as a protein or RNA, e.g., microRNA. As one example, a regulatory sequence can control transcription of a downstream DNA sequence into RNA and thence into a protein; in general, any part of this process may be controlled. As another example the DNA may be recognized by, and bind to, a protein, with the result that (a different part of) the protein begins, or ceases, a function or activity.

Implementations of the techniques can be used to compare an input biological sequence that comprises a DNA sequence with an unknown regulatory function with other DNA sequences that are known to have a similar regulatory function and can make a prediction of whether or not the DNA sequence has the regulatory function. As one example such a regulatory function can be to activate, deactivate, or change the expression level of a gene, RNA, or molecule up or down, e.g., in a particular cell type.

### Physical Synthesis

In general, one or more sequences selected by a sequence selection or identification method, or by a screening process as described herein, can be physically synthesized. This can involve physically synthesizing the sequence or obtaining a physical embodiment of the sequence from a third party. The physical sequence may be in the form of a molecule, e.g., in the case of an amino acid sequence, or may be further processed to obtain a molecule, e.g., in the case of a DNA or RNA sequence. Optionally the structure or function of the sequence can then be investigated in vitro or in vivo to confirm a desired structure or function of the sequence or of a corresponding or related molecule, e.g., efficacy as a drug, or as a vaccine, or as a regulatory or other motif.

### BRIEF DESCRIPTION OF THE DRAWINGS

FIG. 1 is a diagram of an example label identification system.

FIG. 2 is an example scoring engine that can be included in a label identification system.

FIG. 3 is a block diagram illustrating an example training process for training a neural network that can be included in a label identification system.

FIG. 4 is a flow diagram of an example process for identifying labels associated with an input biological sequence.

FIG. 5 is a flow diagram of an example process for identifying a plurality of positive biological sequences associated with a candidate label.

FIG. 6 is a flow diagram of an example process for training a neural network.

FIG. 7 is a flow diagram of an example process for selecting a plurality of candidate labels for an input biological sequence.

FIG. 8 is a flow diagram of an example process for identifying ligands predicted to bind to a molecule including an input biological sequence.

FIG. 9 is a flow diagram of an example process for identifying candidate target molecules to which a molecule including an input biological sequence is predicted to bind.

Like reference numbers and designations in the various drawings indicate like elements.

### DETAILED DESCRIPTION

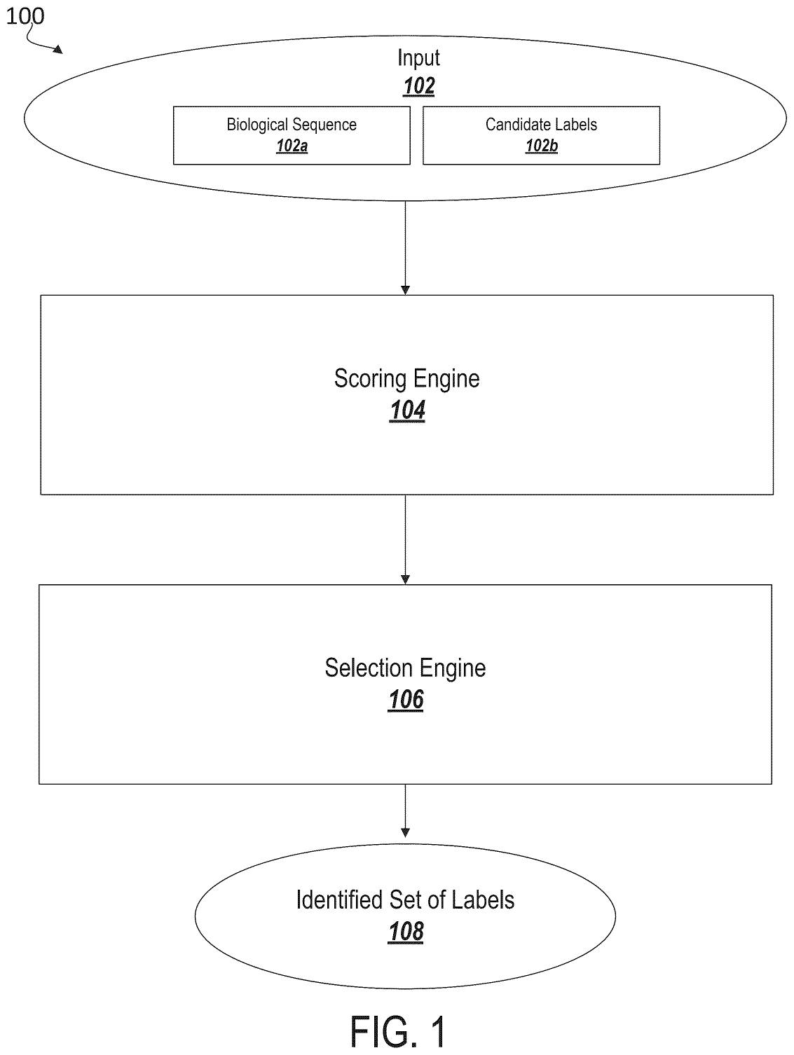

FIG. 1 is a diagram of an example label identification system 100. The label identification system 100 is an example of a system implemented as computer programs on one or more computers in one or more locations, in which the systems, components, and techniques described below can be implemented. The label identification system 100 includes a scoring engine 104 and a selection engine 106.

The label identification system 100 is a system that identifies one or more labels associated with biological sequences. The biological sequences can be sequences of elements included in a biological molecule or structure. For example, a biological sequence can be a nucleotide sequence of a deoxyribonucleic acid (DNA) molecule, a nucleotide sequence of a ribonucleic acid (RNA) molecule, or an amino acid sequence of a protein. An identified label can be any suitable label for a biological sequence. For example, an identified label can specify a biological function of the biological sequence, or of a molecule including the biological sequence.

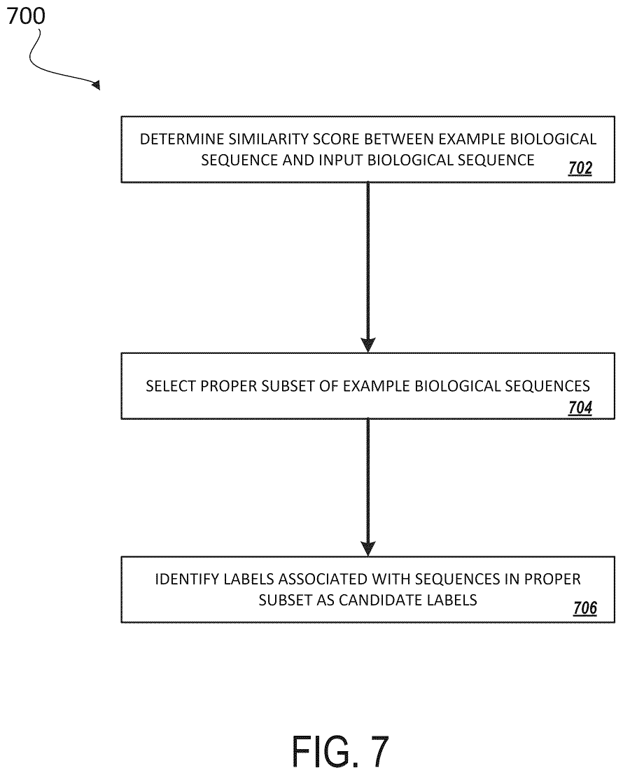

The label identification system 100 receives an input 102 that includes a biological sequence 102a and a set of candidate labels 102b. The biological sequence 102a can be a biological sequence indicated in a request received by the system to identify one or more labels associated with the biological sequence. In some implementations, the label identification system 100 can select the set of candidate labels 102b as a proper subset of a set of possible labels for the biological sequence 102, e.g., according to the techniques described with reference to FIG. 7.

The scoring engine 104 is configured to process the input 102 to generate, for each of the set of candidate labels 102b, a score characterizing a likelihood that the biological sequence 102a is associated with the candidate label. The scoring engine 104 includes a neural network. The neural network can have any suitable architecture. For example, the neural network can have any of a variety of Transformer-based neural network architectures, e.g., encoder-only Transformer architectures, encoder-decoder Transformer architectures, decoder-only Transformer architectures, other attention-based architectures, and so on. The neural network can include any appropriate types of neural network layers (e.g., fully connected layers, convolutional layers, attention layers, and/or recurrent layers) in any appropriate number (e.g., 5 layers, or 10 layers, or 50 layers) and connected in any appropriate configuration (e.g., as a directed graph of layers). For example, the neural network can have a similar architecture to the model described in Raffel et. al., “Exploring the limits of transfer learning with a unified text-to-text transformer.” Journal of machine learning research, 21(140):1-67, 2020.

The neural network has been trained to perform a training task including generating, for each of a set of candidate labels, a score characterizing a likelihood that an input biological sequence is associated with the candidate label. The training of the neural network can include determining gradients of an objective function with respect to current values of a set of neural network parameters of the neural network, wherein the objective function measures an error between: (i) the score generated by the neural network that characterizes the likelihood that an input biological sequence is associated with a given candidate label, and (ii) a target score characterizing the likelihood that the input biological sequence is associated with the given candidate label; and updating the current values of the set of neural network parameters of the neural network using the gradients.

The gradients used for training the neural network can be computed using backpropagation. The gradients can be used to update the parameter values of the neural network using an update rule of any appropriate gradient descent optimization algorithm, e.g., RMSprop or Adam.

The objective function used for training the neural network can be any appropriate objective function. For example, the objective function can measure the error between the generated score and the target score using squared error or absolute error.

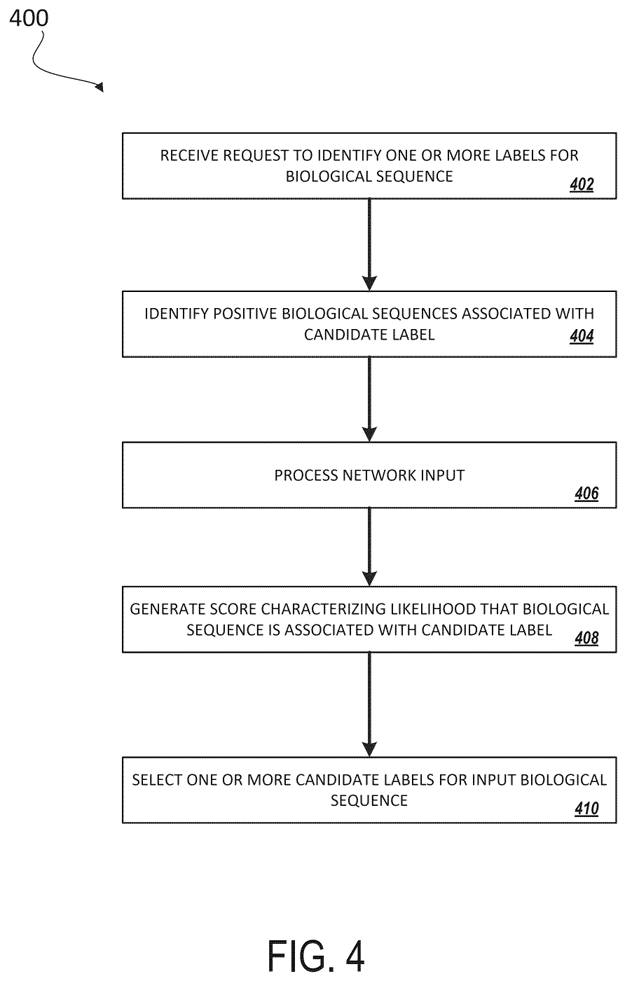

The scoring engine 104 processes the input 102 using the neural network. The score for each of the set of candidate labels 102b characterizing the likelihood that the biological sequence 102a is associated with the candidate label can be represented as a quantitative representation of a probability, as described with reference to FIG. 4.

The selection engine 106 is configured to select one or more candidate labels from the set of candidate labels 102b based on the scores generated by the scoring engine 104. The selection engine selects the one or more candidate labels as labels for the biological sequence 102a. For example, the selection engine 106 can select the one or more candidate labels using the techniques described below with reference to FIG. 4.

In response to receiving the input 102, the label identification system 100 generates as output an identified set of labels 108. The identified set of labels 108 is a subset of the set of candidate labels 102b. The identified set of labels 108 is defined by the one or more candidate labels selected by the selection engine 106, as described above.

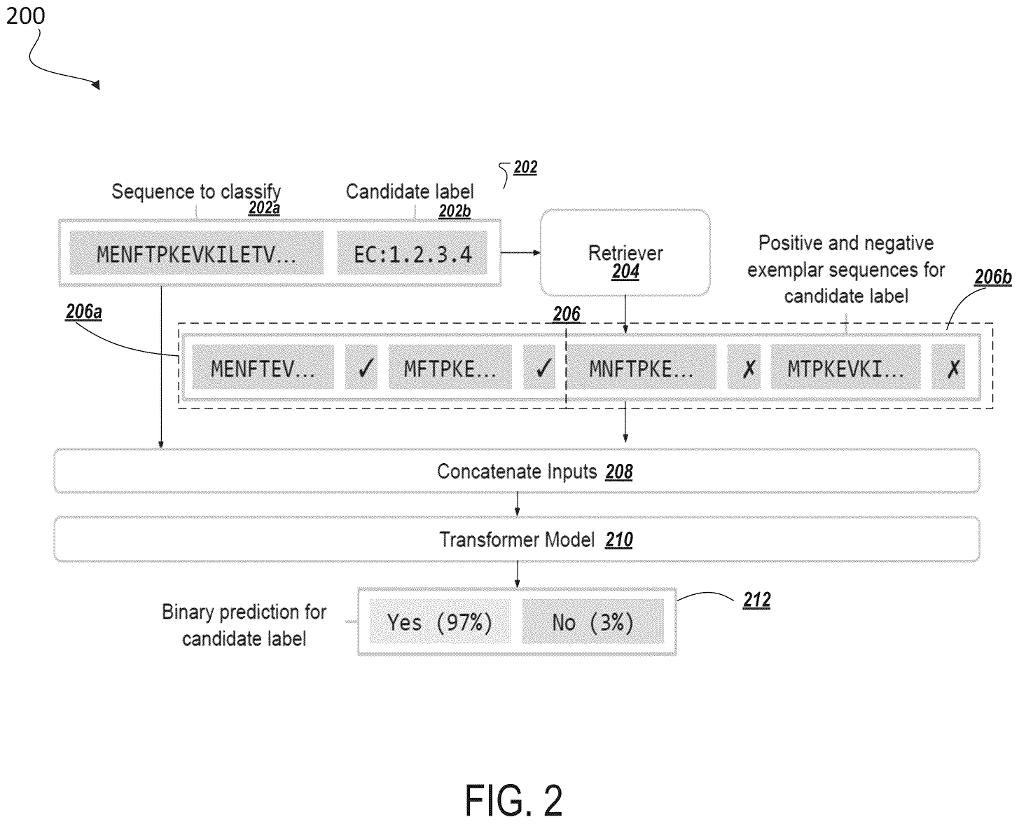

FIG. 2 is an example scoring engine 200. The scoring engine 200 is an example of a system implemented as computer programs on one or more computers in one or more locations, in which the systems, components, and techniques described below can be implemented. The scoring engine 200 includes a retriever 204, an input concatenation engine 208, and a transformer model 210.

The scoring engine is an engine that generates scores characterizing likelihoods that a given biological sequence is associated with a given candidate label. For example, the scoring engine 200 receives an input 202 that includes a biological sequence 202a and a candidate label 202b. The biological sequence 202a can be a sequence of elements included in a biological molecule or structure and can be indicated in a request to identify one or more labels associated with the biological sequence that is received by a system including the scoring engine 200. For example, the biological sequence 202a can be similar to the biological sequence 102a described with reference to FIG. 1. The candidate label 202b can specify a biological function of the biological sequence, or of a molecule including the biological sequence. For example, the candidate label 202b can be similar to any of the candidate labels included in the set of candidate labels 102b of FIG. 1. In some implementations, a system including the scoring engine 200 can receive as input the biological sequence 202a and a set of candidate labels. For each candidate label in the input to the system, the scoring engine 200 can process an input, such as the input 202, that includes the biological sequence 202a and the candidate label.

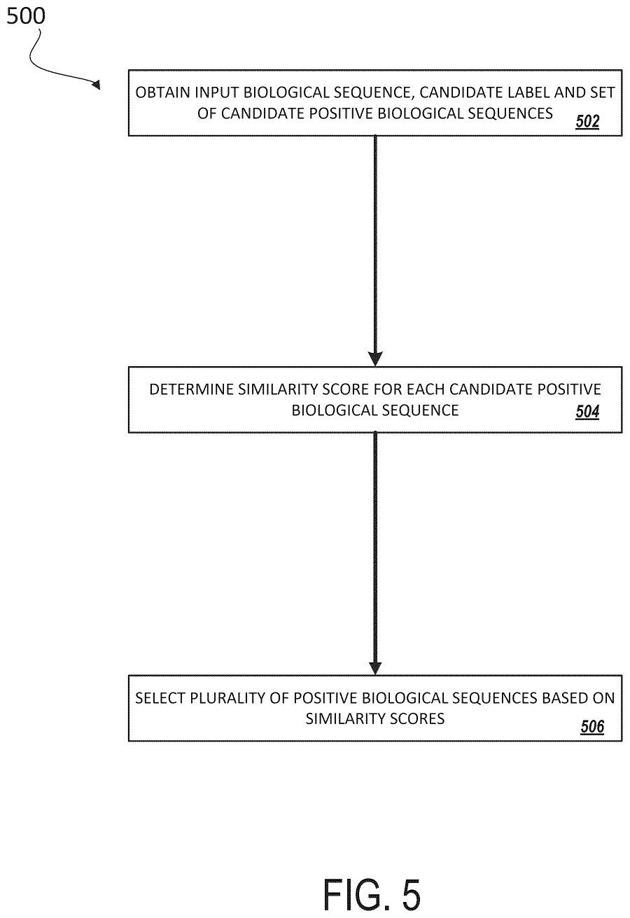

The retriever 204 is configured to process the biological sequence 202a and the candidate label 202b to identify a set of positive biological sequences that are each associated with the candidate label 202b. The retriever 204 can identify the set of positive biological sequences such that each positive biological sequence in the set is similar to the biological sequence 202a. For example, the retriever 204 can identify the set of positive biological sequences using similarity scores that are generated for each of a set of candidate positive biological sequences, where the similarity score for each candidate positive biological sequence measures a similarity between the biological sequence 202a and the candidate positive biological sequence. For example, the retriever 204 can identify the set of positive biological sequences using techniques similar to those described with reference to FIG. 5.

In some implementations, the retriever 204 is also configured to identify a set of negative biological sequences that are each not associated with the candidate label 202b. The retriever 204 can identify the set of negative biological sequences such that each negative biological sequence in the set is similar to the biological sequence 202a, e.g., by using similarity scores such as those used to identify the set of positive biological sequences. For example, the retriever 204 can identify the set of negative biological sequences using techniques similar to those described with reference to FIG. 5.

Thus, the retriever 204 can generate as output a set of biological sequences 206. As described above, the set of biological sequences 206 includes a set of positive biological sequences 206a that are each associated with the candidate label 202b. As described above, in some implementations, the set of biological sequences 206 can also include a set of negative biological sequences 206b that are each not associated with the candidate label 202b.

The input concatenation engine 208 receives the input 202 and the set of biological sequences 206. The input concatenation engine 208 concatenates the input 202 and the set of biological sequences 206 to generate a concatenated output that is to be processed by the transformer model 210.

The transformer model 210 is a neural network with any of a variety of Transformer-based neural network architectures, e.g., encoder-only Transformer architectures, encoder-decoder Transformer architectures, decoder-only Transformer architectures, other attention-based architectures, and so on. For example, the transformer model 210 can be substantially similar to the neural network included in the scoring engine 104 of FIG. 1, as described above.

The transformer model 210 is configured to process the concatenated output, which includes the input 202 and the set of biological sequences 206, to generate a score 212 characterizing the likelihood that the biological sequence 202a is associated with the candidate label 202b. The score 212 can be a quantitative representation of a probability that the biological sequence 202a is associated with the candidate label 202b, as described with reference to FIG. 4. In the example of FIG. 2, the score 212 represents that probability that the biological sequence 202a is associated with the candidate label 202b as a percentage. For example, the score 212 indicates that there is a 97% chance that the biological sequence 202a is associated with the candidate label 202b, and likewise a 3% chance that the biological sequence 202a is not associated with the candidate label 202b.

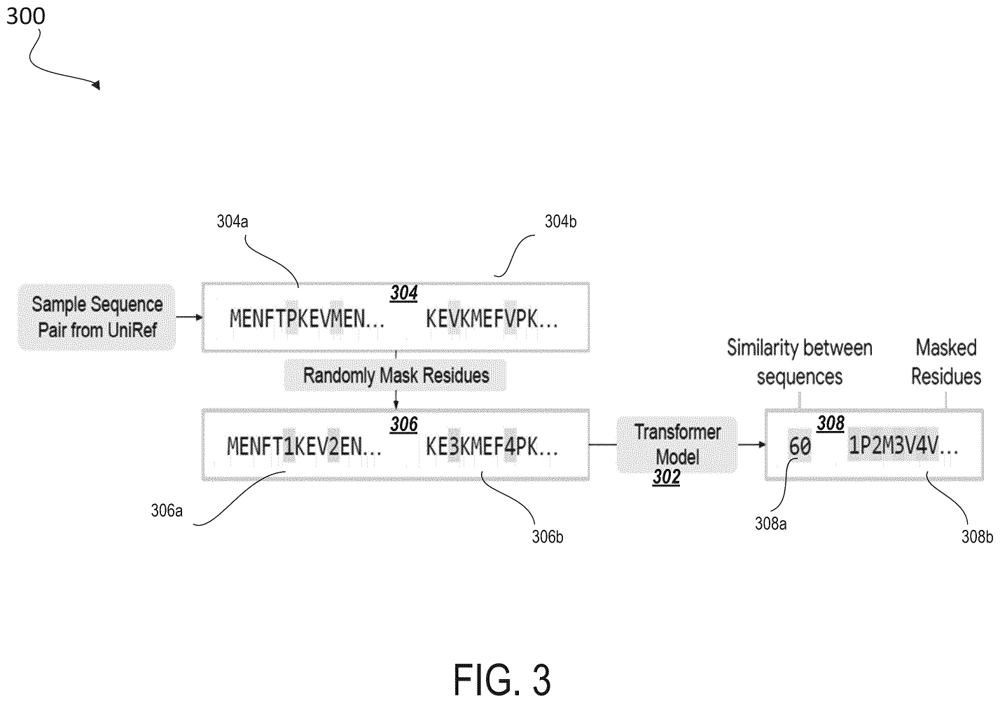

FIG. 3 is a block diagram illustrating an example training process 300 for training a neural network. For convenience, the training process 300 will be described as being performed by a system of one or more computers located in one or more locations. For example, a label identification system, e.g., the label identification system 100 depicted in FIG. 1, appropriately programmed in accordance with this specification, can perform the training process 300. The system can include the neural network 302 trained using the training process 300. The neural network 302 can be a neural network with any of a variety of Transformer-based neural network architectures, e.g., encoder-only Transformer architectures, encoder-decoder Transformer architectures, decoder-only Transformer architectures, other attention-based architectures, and so on. For example, the transformer model 302 can be substantially similar to the neural network included in the scoring engine 104 of FIG. 1, as described above. The transformer model 302 trained according to the training process 300 can be used to identify labels associated with an input biological sequence, e.g., in the manner described with reference to FIG. 4.

The system performing the training process 300 first obtains a pair of training biological sequences 304. The training biological sequences included in the pair 304 can be of the same type as the input biological sequence for which the transformer model 302 is trained to identify associated labels. In the example of FIG. 3, each of the training biological sequences 304 is an amino acid sequence of a protein.

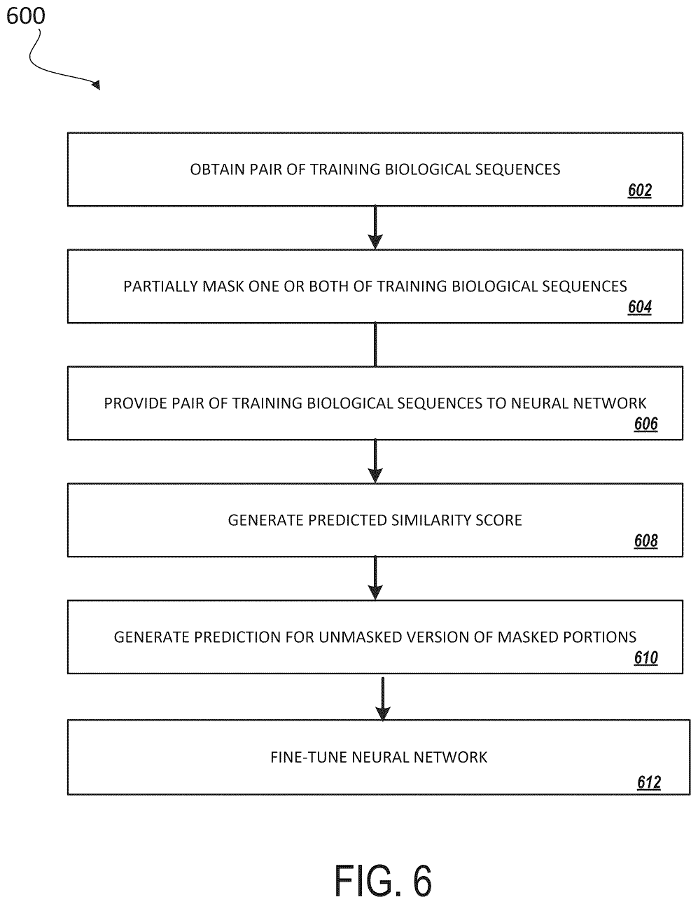

For each biological sequence included in the pair 304, the system randomly masks elements included in the biological sequence to generate a masked representation 306 of the biological sequence. The system can mask any suitable number of elements of the biological sequence in generating the masked representation 306, as described with reference to FIG. 6.

In the example of FIG. 3, the system randomly masks amino acid residues included in each of the amino acid sequences of the pair 304. In the example of FIG. 3, for each biological sequence, the masking is represented by the replacement of a letter of the alphabet representing an amino acid residue included in the biological sequence with a number in the corresponding masked representation 306 of the biological sequence. For example, in the biological sequence 304a of the pair 304, the amino acid residues represented by the highlighted letters “P” and “M” are replaced with the highlighted numbers “1” and “2”, respectively, in the masked representation 306a of the biological sequence 304a. In the biological sequence 304b of the pair 304, the amino acid residues represented by the highlighted letters “V” (e.g., the first ‘V’ in the sequence) and “V” (e.g., the second ‘V’ in the sequence) are replaced with the highlighted numbers “3” and “4”, respectively, in the masked representation 306b of the biological sequence 304b.

The transformer model 302 processes the masked representation 306 of the pair 304 to generate the output 308. The output 308 includes a similarity score 308a and a prediction 308b for an unmasked version of any masked portions of the pair 304 of training biological sequences. The similarity score 308a can be any suitable measure of similarity between the biological sequences of the pair 304. For example, the similarity score can be determined using a Levenshtein distance between the biological sequences of the pair 304, e.g., according to the techniques described with reference to FIG. 6.

The prediction 308b can include, for each training biological sequence that the system partially masks, for each element of the sequence that the system masks, a prediction for an unmasked version of the element, e.g., as described with reference to FIG. 6. In the example of FIG. 3, the prediction 308b includes a prediction of “P” as the unmasked version of the masked element represented by a “1” in the masked representation 306a; a prediction of “M” as the unmasked version of the masked element represented by a “2” in the masked representation 306a; a prediction of “V” as the unmasked version of the masked element represented by a “3” in the masked representation 306b; and a prediction of “V” as the unmasked version of the masked element represented by a “4” in the masked representation 306b.

The training process 300 can be repeated multiple times for each of a training set of pairs of training biological sequences, of which the pair 304 is one example. By repeatedly performing the process 300, the system can train the transformer model 302 to optimize an objective function, e.g., according to the techniques described with reference to FIG. 6. In some implementations, the system can train the transformer model 302 to optimize the objective function by, after repeating the training process 300 a suitable number of times, fine-tuning the transformer model 302, e.g., according to the techniques described with reference to the operation 610 of FIG. 6.

In implementations in which the transformer model 302 is used to identify labels associated with an input biological sequence, training the transformer model 302 to optimize an objective function in this way can cause the transformer model 302 to implicitly compare input biological sequences with candidate sequences that are or are not associated with a given candidate label. The transformer model can use the comparison of the input biological sequences with the candidate sequences to determine whether to identify the label as associated with the input biological sequence, improving the performance of the transformer model, as described above.

FIG. 4 is a flow diagram of an example process 400 for identifying labels associated with an input biological sequence. For convenience, the process 400 will be described as being performed by a system of one or more computers located in one or more locations. For example, a label identification system, e.g., the label identification system 100 depicted in FIG. 1, appropriately programmed in accordance with this specification, can perform the process 400.

The system receives a request to identify one or more labels associated with an input biological sequence (402). The input biological sequence can be a sequence of elements included in a biological molecule or structure, e.g., any of the biological sequences described with reference to FIG. 1.

In response to receiving the request, for each of a plurality of candidate labels, the system identifies a plurality of positive biological sequences that are each associated with the candidate label (404). The system can identify the plurality of positive biological sequences using techniques similar to those described with reference to FIG. 5. In some implementations, the system also identifies, for each of the plurality of candidate labels, a plurality of negative biological sequences that are each not associated with the candidate label. The system can identify the plurality of negative biological sequences using techniques similar to those described with reference to FIG. 5.

The plurality of candidate labels can include any suitable types of labels for biological sequences. For example, in some implementations, the plurality of candidate labels can include one or more biological function labels that each specify a respective biological function. In such implementations, a biological sequence can be associated with a biological function label if molecules including the biological sequence have the biological function specified by the biological function label.

In implementations in which the input biological sequence is an amino acid sequence of a protein, the plurality of candidate labels can include one or more subcellular localization labels that each specify a subcellular location. In such implementations, a biological sequence can be associated with a subcellular localization label if molecules including the biological sequence are active in the subcellular location specified by the subcellular localization label.

In implementations in which the input biological sequence is an amino acid sequence of a protein, the plurality of candidate labels can include one or more enzymatic activity labels that each specify a respective type of reaction. In such implementations, a biological sequence is associated with an enzymatic activity label if the molecules including the biological sequence are involved in catalyzing the type of reaction specified by the enzymatic activity label.

In implementations in which the input biological sequence is an amino acid sequence of a protein, the plurality of candidate labels can include a solubility label. In such implementations, a biological sequence is associated with the solubility label if molecules including the biological sequence are soluble.

In some implementations, the system selects the plurality of candidate labels as a proper subset of a set of possible labels for the input biological sequence. A proper subset of the set of possible labels can be a subset of one or more labels such that each label in the subset is in the set of possible labels, and such that there is at least one label in the set of possible labels that is not included in the subset. For example, the system can select the plurality of candidate labels using techniques similar to those described with reference to FIG. 7.

For each of the plurality of candidate labels, the system processes a network input (406). The network input includes: (i) the input biological sequence, and (ii) the plurality of positive biological sequences that are each associated with the candidate label. In some implementations, the network input also includes data identifying the candidate label. In implementations in which the system identifies a plurality of negative biological sequences, the network input also includes the plurality of negative biological sequences that are each not associated with the candidate label. The biological sequences included in the network input can each be represented as a sequence of tokens, e.g., a sequence of tokens defining an amino acid sequence or a nucleotide sequence or a SMILES string.

In some implementations, the network input also includes labeling data that identifies each of the plurality of positive biological sequences as being associated with the candidate label, and each of the plurality of negative biological sequences as not being associated with the candidate label. For example, the labeling data can be represented as a respective one or more tokens for each of the biological sequences included in the network input. For example, the respective one or more tokens can represent a binary identifier. For example, the binary identifier can have one of two values: either a “positive” value to indicate that the biological sequence is associated with the candidate label, or a “negative” value to indicate that the biological sequence is not associated with the candidate label.

In some implementations, the network input also includes data characterizing a three-dimensional (3D) structure of a molecule that includes the input biological sequence. In some implementations, the network input also includes, for one or more of the plurality of positive biological sequences, data characterizing a respective 3D structure of a molecule that includes the positive biological sequence. In implementations in which the network input includes a plurality of negative biological sequences, the network input can also include for one or more of the plurality of negative biological sequences, data characterizing a respective 3D structure of a molecule that includes the negative biological sequence.

In some implementations, the network input can be represented as a sequence of embeddings. In such implementations, processing the network input can include processing the representation of the network input as a sequence of embeddings using a neural network.

The system processes the network input using a neural network and in accordance with values of a set of neural network parameters. The neural network can have any suitable architecture. For example, the neural network can have any of the example architectures described with reference to the neural network included in the scoring engine 104 of FIG. 1.

For each of the plurality of candidate labels, the system generates a score characterizing the likelihood that the input biological sequence is associated with the candidate label (408). The score can be a quantitative representation of a probability that the input biological sequence is associated with the candidate label. For example, the probability can be a percentage, a ratio, a number between 0 and 1, or any other suitable representation of a probability.

The system selects one or more candidate labels of the plurality of candidate labels as labels for the input biological sequence based on the scores (410). For example, the system can select the one or more candidate labels by selecting the candidate labels with scores that characterize high likelihoods that the input biological sequence is associated with the candidate label. For example, the system can select the one or more candidate labels such that, for each selected candidate label, the likelihood characterized by the score of the candidate label is higher than each of the likelihoods characterized by the scores of the candidate labels that are not selected.

In some implementations, the system can select N candidate labels of the plurality of candidate labels, where Nis an integer. For example, the system can select the N candidate labels to be the N candidate labels of the plurality of candidate labels with scores that characterize the highest likelihoods of the likelihoods characterized by all of the scores for the candidate labels of the plurality. In some implementations, the system can select the one or more candidate labels to be all candidate labels of the plurality with scores that exceed a score threshold. For example, the score threshold can be determined such that candidate labels with scores that exceed the score threshold have at least a threshold likelihood of being associated with the input biological sequence, e.g., a threshold likelihood that can be determined by a user of the system.

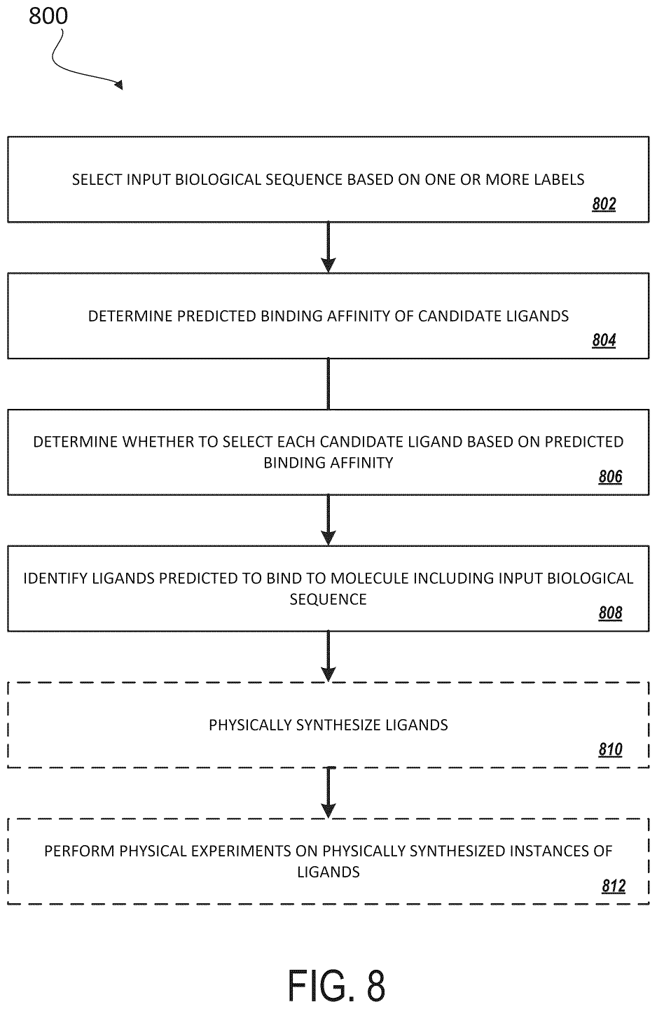

In some implementations, the system selects the input biological sequence as a drug target or substrate of an industrial enzyme based at least in part on the one or more labels selected for the input biological sequence. In such implementations, the system can identify one or more ligands that are predicted to bind to a molecule that includes the input biological sequence, e.g., according to the techniques described with reference to FIG. 8.

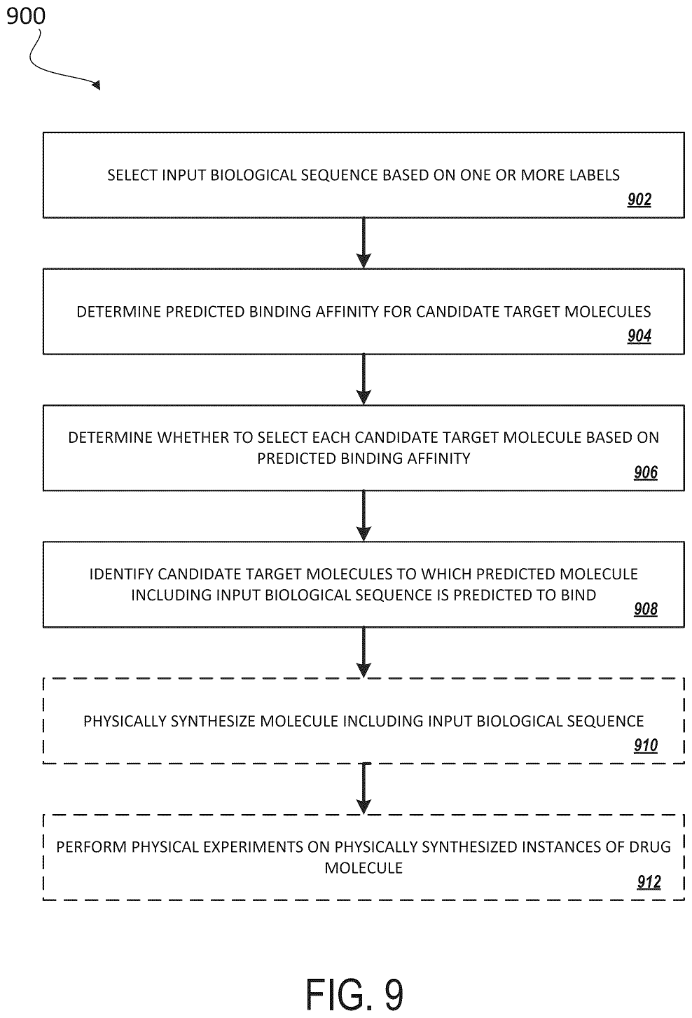

In some implementations, the system selects the input biological sequence for inclusion in a molecule or selects a molecule that is otherwise defined by the input biological sequence, based at least in part on the one or more labels selected for the input biological sequence. In such implementations, the system can identify one or more candidate target molecules to which a molecule that includes the input biological sequence is predicted to bind, the molecule that includes the input biological sequence being a drug molecule or an industrial enzyme and each candidate target molecule being a candidate target molecule of the drug molecule or a candidate substrate molecule of the industrial enzyme. For example, the system can identify the one or more candidate target molecules using techniques such as those described with reference to FIG. 9.

In some implementations, the system uses the process 400 for identifying the presence of one or more diseases and the input biological sequence is determined by analyzing a version of a protein or nucleic acid obtained from a human or animal body. For example, the one or more diseases can include at least one of a genetic disease; a protein mis-folding disease; and a nucleic acid mis-folding disease. In such implementations, the system can determine that the input biological sequence is associated with one or more diseases based at least in part on the one or more labels selected for the input biological sequence.

In implementations in which the input biological sequence includes an amino acid sequence of a protein, the system can select the protein as a target for increasing or decreasing production of an output produced by a biochemical pathway based at least in part on the one or more labels selected for the input biological sequence of the protein. The system can determine one or more genetic edits to modulate expression of the protein. In some implementations, the system can apply the one or more genetic edits to a genome of an organism to modulate the expression of the protein.

In some implementations, the process 400 is for identifying the presence of one or more diseases and the input biological sequence is determined by analyzing a version of a protein or nucleic acid obtained from a human or animal body. In such implementations, the system also determines that the input biological sequence is associated with one or more diseases based at least in part on the one or more labels selected for the input biological sequence. In such implementations, the one or more diseases can include at least one of: a genetic disease; a protein mis-folding disease; and a nucleic acid mis-folding disease.

In implementations in which the input biological sequence includes an amino acid sequence of a protein, the system also selects the protein as a target for increasing or decreasing production of an output produced by a biochemical pathway based at least in part on the one or more labels selected for the input biological sequence of the protein. The system then determines one or more genetic edits to modulate expression of the protein. In such implementations, the system can also apply the one or more genetic edits to a genome of an organism to modulate the expression of the protein.

FIG. 5 is a flow diagram of an example process 500 for identifying a plurality of positive biological sequences associated with a candidate label. For convenience, the process 500 will be described as being performed by a system of one or more computers located in one or more locations. For example, a retriever of biological sequences, e.g., the retriever 204 of FIG. 2, appropriately programmed in accordance with this specification, can perform the process 500.

In some implementations, the system can be part of a larger system that identifies labels for biological sequences, e.g., the label identification system 100 of FIG. 1. The system can perform the process 500 for each of a plurality of candidate labels for an input biological sequence. The larger system can then use the positive biological sequences identified for each of the plurality of candidate labels for identifying labels for the input biological sequence.

The system obtains an input biological sequence, a candidate label, and a set of candidate positive biological sequences (502). The input biological sequence can be a biological sequence for which the system is to identify labels. Each candidate positive biological sequence included in the set is associated with the candidate label.

In some implementations, the system obtains the set of candidate positive biological sequences from a training set, e.g., such that the set includes the entire training set or a subset of the training set. The training set can be a set of biological sequences, each biological sequence associated with a label, that can be used to train a neural network included in the system. For example, the training set can be used to train a neural network included in the system to generate a score characterizing the likelihood that an input biological sequence is associated with a candidate label. For example, the training set can be used to train a neural network similar to the transformer model 210 of FIG. 2.

In some implementations, the system obtains the set of candidate positive biological sequences from a collection of biological sequences that all share a particular feature. In some implementations, the collection is included in the training set. For example, the particular feature that is shared by all the biological sequences of the collection can be: a biological function (e.g., in implementations in which the system identifies labels for biological sequences in order to screen biological sequences for biological functions); giving rise to an immune response (e.g., in implementations in which the system identifies labels for biological sequences in order to identify vaccines); being related to a particular motif (e.g., in implementations in which the system identifies labels for biological sequences in order to identify sequences or molecules with the particular motif); a biological function (e.g., in implementations in which the system identifies labels for biological sequences in order to screen biological sequences for biological functions).

The set of candidate positive biological sequences obtained by the system can be included in the training set of biological sequences that is used to train the neural network. For example, the set of candidate positive biological sequences can include all of the biological sequences of the training set that are associated with the candidate label.

In some implementations, the system obtains the set of candidate positive biological sequences by first determining a respective similarity score for each of the candidate positive biological sequences in a larger set of candidate positive biological sequences (e.g., a set of candidate positive biological sequences included in a training set as described above). The similarity score can be similar to the similarity score determined during operation 504, described below.

The system can then rank the candidate positive biological sequences in the larger set according to the similarity scores. For example, the system can assign to each candidate positive biological sequence a rank that is based on the respective similarity score. The system can assign the ranks such that, for each pair of candidate positive biological sequences of the larger set, a first candidate positive biological sequence included in the pair with a similarity score that is higher than that of a second candidate positive biological sequence included in the pair will be assigned a higher rank than the rank assigned to the second candidate positive biological sequence.

The system can obtain the set of candidate positive biological sequences by selecting, from the larger set, a subset of candidate positive biological sequences that have the highest rank. For example, the system can select the subset of highest ranked candidate positive biological sequences from the larger set such that each candidate positive biological sequence included in the selected subset is assigned a rank that is higher than the rank assigned to each of the candidate positive biological sequences not included in the selected subset. The number of candidate positive biological sequences included in the selected subset can be any suitable number, e.g., 100.

In some implementations, the system obtains the set of candidate positive biological sequences by selecting, from a larger set of candidate positive biological sequences (e.g., a set of candidate positive biological sequences included in a training set as described above), a number of candidate positive biological sequences from each of a set of classes of candidate positive biological sequences included in the larger set. A class of sequences can be a group of sequences that all share at least one feature (e.g., a feature related to the labels associated with each of the biological sequences, such as one or more domains, one or more biological functions of molecules in which they are included, one or more subcellular locations in which molecules in which they are included are active, one or more levels of solubility of molecules in which they are included, or one or more types of reactions that molecules in which they are included are involved in catalyzing).

The system can select a same number of candidate positive biological sequences from each class of the set of classes, or the system can select a different number of candidate positive biological sequences from each class. In some implementations, the system can randomly select the number of candidate positive biological sequences from each class. The system can then generate a similarity score for each of the selected candidate positive biological sequences. The similarity score can be similar to the similarity score determined during operation 504, described below. The system can then rank the selected candidate positive biological sequences according to their similarity scores, as described above.

This technique for obtaining the set of candidate positive biological sequences can cause the positive biological sequences identified by the system via the process 500 to be more useful in identifying labels for the input biological sequence. For example, the system can include the positive biological sequences in a network input to a neural network that generates a score characterizing the likelihood that the input biological sequence is associated with the candidate label and identify labels for the input biological sequence using the score. The positive biological sequences identified using this technique can represent a larger variety of classes of biological sequences, which can enable the neural network to generate a score that is more likely to accurately characterize the likelihood that the input biological sequence is associated with the candidate label.

The system determines, for each candidate positive biological sequence in the set, a respective similarity score (504). The respective similarity score for each candidate positive biological sequence measures a similarity between: (i) the candidate positive biological sequence, and (ii) the input biological sequence. The respective similarity score for each candidate positive biological sequence can be represented as any suitable measure of similarity. For example, each respective similarity score can be a quantitative measure of the similarity between the candidate positive biological sequence and the input biological sequence, e.g., a percentage value indicating a percentage of the candidate positive biological sequence that matches or is similar to the input biological sequence. For example, each respective similarity score can be a quantitative measure of an extent to which the elements of the candidate positive biological sequence align with corresponding elements of the input biological sequence.

In some implementations, each respective similarity score can be determined by identifying and scoring local sequence alignments in each of the candidate positive biological sequence and the input biological sequence. In some implementations, each respective similarity score can be determined using an empirically-derived substitution cost matrix. For example, each respective similarity score can be determined using techniques such as those described in Altschul et. al., “Basic local alignment search tool.” Journal of molecular biology, 215(3):403-410,1990; Altschul et. al., “Gapped blast and psi-blast: a new generation of protein database search programs.” Nucleic acids research, 25(17):3389-3402, 1997; or Henikoff and Henikoff, “Amino acid substitution matrices from protein blocks.” Proceedings of the National Academy of Sciences, 89(22):10915-10919, 1992.

In some implementations, upon determining a respective similarity score for each of the candidate positive biological sequences included in the set, the system ranks the candidate positive biological sequences according to the determined similarity scores, as described above.

In implementations in which the system obtains the set of candidate positive biological sequences by first determining a respective similarity score for each of the candidate positive biological sequences in a larger set of candidate positive biological sequences, the system can determine the respective similarity score for each candidate positive biological sequence to be the similarity score determined by the system for the candidate positive biological sequence in obtaining the set of candidate positive biological sequences, as described above.

In implementations in which the system obtains the set of candidate positive biological sequences by selecting a number of candidate positive biological sequences from each of a set of classes of candidate positive biological sequences included in a larger set, the system can determine the respective similarity score for each candidate positive biological sequence to be the similarity score determined by the system in obtaining the set of candidate positive biological sequences, as described above.

The system selects the plurality of positive biological sequences based on the similarity scores (506). The system selects the plurality of positive biological sequences as a proper subset of the set of candidate positive biological sequences (e.g., each positive biological sequence in the proper subset is included in the set of candidate positive biological sequences, and there is at least one candidate positive biological sequence in the set that is not included in the proper subset).

In some implementations, the system selects the plurality of positive biological sequences by selecting a plurality of highest ranked candidate positive biological sequences under a ranking of the set of candidate positive biological sequences based on the similarity scores. The ranking of the set of candidate positive biological sequences can be a ranking determined using techniques similar to those used in the operation 502, described above. Each candidate positive biological sequence of the set can be assigned a rank according to the ranking. For example, the system can select the plurality of highest ranked candidate positive biological sequences such that each candidate positive biological sequence included in the selected plurality is assigned a rank that is higher than the rank assigned to each of the candidate positive biological sequences not included in the selected plurality.

In some implementations, the system selects the plurality of positive biological sequences by stochastically sampling the plurality of positive biological sequences from the set of candidate positive biological sequences based on the similarity scores. For example, the system can select the plurality of positive biological sequences from the set of candidate positive biological sequences according to a probability distribution. In some implementations, the probability distribution can depend on the similarity scores. For example, the system can select the plurality of positive biological sequences by uniformly sampling from the set of candidate positive biological sequences. For example, the system can select the plurality of positive biological sequences by sampling from the set of candidate positive biological sequences according to a distribution given by:

$$P ∝ {p ( 1 - p )}^{j - 1}$$

Where P is the probability that the sequence assigned the jth rank is sampled, and p is a parameter of the distribution.

In some implementations, the system uses a process similar to the process 500 to identify a plurality of negative biological sequences that are each not associated with the candidate label. For example, the system can obtain an input biological sequence, a candidate label, and a set of candidate negative biological sequences using techniques similar to those described above with respect to identifying a plurality of candidate positive biological sequences. For example, the system can obtain the set of candidate negative biological sequences from a training set such that the set of candidate negative biological sequences includes all the biological sequences of the training set that are not associated with the candidate label.

The system can determine a respective similarity score for each candidate negative biological sequence in the set using techniques similar to those described above with respect to identifying a plurality of candidate positive biological sequences. The system can select the plurality of negative biological sequences as a proper subset of the set of candidate negative biological sequences based on the similarity scores, using techniques similar to those described above with respect to identifying a plurality of candidate positive biological sequences.

FIG. 6 is a flow diagram of an example process 600 for training a neural network. For convenience, the process 600 will be described as being performed by a system of one or more computers located in one or more locations. For example, a label identification system, e.g., the label identification system 100 depicted in FIG. 1, appropriately programmed in accordance with this specification, can perform the process 600. The system can include the neural network trained using the process 600. The neural network trained according to the process 600 can be used to identify labels associated with an input biological sequence, e.g., in the manner described with reference to FIG. 4. For example, in some implementations, the neural network can be the transformer model 210 of FIG. 2.

The system obtains a pair of training biological sequences (602). Each of the training biological sequences can be a sequence of elements representing a biological molecule or structure. For example, each of the training biological sequences can be a nucleotide sequence of a DNA molecule, a nucleotide sequence of a RNA molecule, or an amino acid sequence of a protein. In implementations in which the neural network is used to identify labels associated with an input biological sequence, each of the training biological sequences can be of the same type as the input biological sequence.

The system partially masks one or both training biological sequences of the pair of training biological sequences (604). For each of the one or both training biological sequences that the system partially masks, partially masking the training biological sequence can include generating a masked representation of the training biological sequence by randomly masking a number of elements of the sequence. The number of elements of the sequence that are masked can be any suitable number, e.g., one element or any integer number of elements where the integer number is less than the total number of elements included in the sequence. For example, the number of elements in the sequence that are masked can be equal to 10% of the number of elements in the sequence.

The system provides the pair of training biological sequences as a network input to the neural network (606). For each training biological sequence that the system partially masks, the system can provide the masked representation of the training biological sequence as a network input to the neural network, e.g., in place of an unmasked version of the training biological sequence.

The system processes the network input to generate a predicted similarity score that is a prediction for a similarity between the pair of input biological sequences (608). The system processes the network input using the neural network. The similarity score can be any suitable measure of similarity between the input biological sequences. For example, the similarity score can be determined using a Levenshtein distance between the input biological sequences. In some implementations, the Levenshtein distance used in determining the similarity score can be normalized by the length of each of the input biological sequences. In some implementations, the Levenshtein distance used in determining the similarity score can be rounded to a nearest integer multiple, e.g., the nearest multiple of 5 or 10.

The system processes the network input to generate a prediction for an unmasked version of any masked portions of the pair of training biological sequences (610). The system processes the network input using the neural network. For each training biological sequence that the system partially masks, for each element of the sequence that the system masks, the system can generate a prediction for an unmasked version of the element. For example, if the training biological sequence is a sequence of nucleotide bases included in a DNA sequence, the system can generate a predicted nucleotide base for each element of the sequence that the system masks. For example, if the training biological sequence is a sequence of amino acids included in a protein, the system can generate a predicted amino acid for each amino acid of the sequence that the system masks.

The system can perform the operations 602-610 for each of a training set of pairs of biological sequences. In some implementations, the training set of pairs of biological sequences is obtained by sampling pairs of sequences with varying degrees of similarity from a database of biological sequences. For each of the training set of pairs of biological sequences, the system can compute an objective function. The objective function can measure an error between the predicted similarity score generated by the neural network for the pair of biological sequences and a target similarity score for the pair of biological sequences. The objective function can also measure an error, for each of the elements of the pair of biological sequences that the system masks, between (i) the prediction for the unmasked version of the element generated by the neural network, and (ii) the element. The objective function can be any suitable function. For example, the objective function can be a span denoising objective function, e.g., the span denoising objective function described in Raffel et. al., “Exploring the limits of transfer learning with a unified text-to-text transformer.” Journal of machine learning research, 21(140):1-67,2020.

Using the training set of pairs of biological sequences, the system can train the neural network to optimize the objective function. The system can train the neural network to optimize the objective function in any suitable way. For example, the training of the neural network can include determining gradients of the objective function with respect to current values of a set of neural network parameters of the neural network and updating the current values of the set of neural network parameters of the neural network using the gradients.

The gradients used for training the neural network can be computed using backpropagation. The gradients can be used to update the parameter values of the neural network using an update rule of any appropriate gradient descent optimization algorithm, e.g., RMSprop or Adam.

The system fine-tunes the neural network to perform a fine-tuning task of predicting labels for training biological sequences (612). The system can fine-tune the neural network using a set of training examples, where each training example in the set includes a biological sequence; a target set of predicted labels for the biological sequence; and, for each of one or more predicted labels in the target set of predicted labels, a positive biological sequence that is associated with the predicted label. In some implementations, each training example can also include, for each of the one or more predicted labels in the target set of predicted labels, a negative biological sequence that is not associated with the predicted label.

The system can train the neural network to optimize an objective function. For a given training example, the objective function can measure an error between: i) a set of predicted labels for the biological sequence included in the training example that is generated by the neural network, and ii) the target set of predicted labels included in the training example.

The neural network can be trained to optimize the objective function in any suitable way. For example, the training of the neural network can include techniques substantially similar to those used for training the neural network with respect to the operations 602-610, as described above.

In some implementations, the process 600 can include additional operations, fewer operations, or some of the operations can be divided into multiple operations. For instance, the process can include operations 602, 606, 608, and 612, optionally including operations 604 and 610. In some implementations, the process 600 for training a neural network can be characterized as including two stages. For example, the first stage can be characterized as a “pre-training” stage and include the operations 602-610. For example, the second stage can be characterized as a “fine-tuning” stage and include the operation 612. As described above, training a neural network using the process 600 can cause the neural network to use a comparison of an input biological sequence with candidate sequences in identifying labels associated with the input biological sequence.

FIG. 7 is a flow diagram of an example process 700 for selecting a plurality of candidate labels for an input biological sequence. For convenience, the process 700 will be described as being performed by a system of one or more computers located in one or more locations. For example, a label identification system, e.g., the label identification system 100 depicted in FIG. 1, appropriately programmed in accordance with this specification, can perform the process 700.

The system can perform the process 700 in response to receiving a request to identify one or more labels associated with an input biological sequence. For example, the system can perform the process 700 as part of the process 400, e.g., after the operation 402 and before the operation 404 of the process 400. For example, the plurality of candidate labels selected via the process 700 can be the plurality of candidate labels for each of which a system performing the process 400 identifies a plurality of associated positive biological sequences at the operation 404.

The system determines, for each example biological sequence in a set of example biological sequences, a respective similarity score (702). For each example biological sequence, the respective similarity score measures a similarity between: (i) the example biological sequence, and (ii) the input biological sequence. The similarity score can be any suitable measure of similarity between the example biological sequence and the input biological sequence. For example, the similarity score can be similar to the similarity score described with reference to FIG. 5.

The set of example biological sequences can be a training set of biological sequences. The training set can be a set of biological sequences, each biological sequence associated with a label, that can be used to train a neural network included in the system. For example, the training set can be used to train a neural network included in the system to generate a score characterizing the likelihood that an input biological sequence is associated with an input candidate label. For example, the training set can be used to train a neural network similar to the transformer model 210 of FIG. 2.

The system selects a proper subset of the set of example biological sequences based on the similarity scores (704). In some implementations, the system selects the proper subset of the set of example biological sequences by selecting a plurality of highest ranked example biological sequences under a ranking of the set of example biological sequences based on the similarity scores.

For example, the system can rank the example biological sequences in the set by assigning to each example biological sequence a rank that is based on the respective similarity score. The system can assign the ranks such that, for each pair of example biological sequences in the set, a first example biological sequence included in the pair with a similarity score that is higher than that of a second example biological sequence included in the pair will be assigned a higher rank than the rank assigned to the second example biological sequence.

The system can then select the plurality of highest ranked example biological sequences by selecting the plurality such that each example biological sequence included in the selected plurality is assigned a rank that is higher than the rank assigned to each of the example biological sequences not included in the selected plurality. The number of example biological sequences included in the selected plurality can be any suitable number, e.g., 100.

The system identifies each label that is associated with at least one of the example biological sequences in the selected proper subset of the set of example biological sequences as a candidate label for the input biological sequence (706). The selected plurality of candidate labels for the input biological sequence can be defined by the labels thus identified by the system.

FIG. 8 is a flow diagram of an example process 800 for identifying ligands predicted to bind to a molecule including an input biological sequence. For convenience, the process 800 will be described as being performed by a system of one or more computers located in one or more locations.

The system selects an input biological sequence based at least in part on one or more labels selected for the input biological sequence (802). The one or more labels can have been selected for the input biological sequence by a label identification system, e.g., the label identification system 100 of FIG. 1. For example, the one or more labels can have been selected for the input biological sequence according to the techniques described with reference to FIG. 4.

The system selects the input biological sequence as a drug target or substrate of an industrial enzyme using the one or more labels. For example, the one or more labels can be labels such that, if associated with a biological sequence, indicate that molecules including the biological sequence are to be targeted by a drug, e.g., a drug that a user of the system desires to manufacture. For example, the one or more labels can be labels such that, if associated with a biological sequence, indicate that molecules including the biological sequence are likely to be targets of an industrial enzyme, e.g., an industrial enzyme that a user of the system desires to manufacture.

The system determines a predicted binding affinity of each candidate ligand in a collection of candidate ligands for a molecule that includes the input biological sequence (804). In some implementations, the molecule including the input biological sequence can include a receptor. In some implementations, the molecule including the input biological sequence can include an antibody or aptamer target including the input biological sequence, such as a virus or cancer cell protein.

As an example, the system can determine the predicted binding affinity of each candidate ligand using a model that is configured to process an input including data representing a ligand and a molecule to generate as output a predicted binding affinity of the ligand for the molecule.

The system determines whether to select each candidate ligand based at least in part on the predicted binding affinity of each candidate ligand for the molecule that includes the input biological sequence (806). The system determines whether to select each candidate ligand as a ligand that is predicted to bind to the molecule that includes the input biological sequence.

For example, the system can determine, for each candidate ligand, to select the candidate ligand if the predicted binding affinity of the candidate ligand for the molecule is above a binding affinity threshold. For example, the system can determine to select candidate ligands that have the highest predicted binding affinities of the candidate ligands in the collection of candidate ligands (e.g., each selected candidate ligand has a higher predicted binding affinity than each of the candidate ligands in the collection which are not selected). The system can determine to select any suitable number of candidate ligands.

The system identifies one or more ligands that are predicted to bind to the molecule that includes the input biological sequence (808). The system can identify the one or more ligands to be the candidate ligands that the system has determined to select as ligands that are predicted to bind to the molecule that includes the input biological sequence at the operation 806.

In some implementations, each of the identified one or more ligands can be a polypeptide ligand, a polynucleoside ligand, or a polynucleotide ligand, or an antibody, or an aptamer. In implementations in which the molecule that includes the input biological sequence includes a receptor, the identified one or more ligands can be agonists or antagonists of the receptor. In implementations in which the molecule that includes the input biological sequence includes an antibody or aptamer target that includes the input biological sequence, the identified one or more ligands can be antibodies or aptamers that bind to the antibody or aptamer target to provide a therapeutic effect.

In some implementations, the system physically synthesizes each of the one or more ligands that are predicted to bind to the molecule that includes the input biological sequence (810).

In some implementations, the system performs, for each of the one or more ligands that are predicted to bind to the molecule that includes the input biological sequence, physical experiments on physically synthesized instances of the ligand (812). The physical experiments are performed by the system to determine one or more of: absorption properties of the ligand, or distribution properties of the ligand, or metabolism properties of the ligand, or excretion properties of the ligand, or toxicity properties of the ligand.

In some implementations, the process 800 can include additional operations, fewer operations, or some of the operations can be divided into multiple operations. For instance, the process can include operations 802, 804, 806, and 808, optionally including operations 810 and 812.

FIG. 9 is a flow diagram of an example process 900 for identifying candidate target molecules to which a molecule including an input biological sequence is predicted to bind. For convenience, the process 900 will be described as being performed by a system of one or more computers located in one or more locations.

The system selects an input biological sequence based at least in part on one or more labels selected for the input biological sequence (902). The one or more labels can have been selected for the input biological sequence by a label identification system, e.g., the label identification system 100 of FIG. 1. For example, the one or more labels can have been selected for the input biological sequence according to the techniques described with reference to FIG. 4.

The system selects the input biological sequence for inclusion in a molecule using the one or more labels. In some implementations, instead of selecting the input biological sequence for inclusion in a molecule, the system selects a molecule that is otherwise defined by the input biological sequence using the one or more labels.

For example, the one or more labels can be labels such that, if associated with a biological sequence, indicate that molecules including the biological sequence, or otherwise defined by the biological sequence, are likely to be drug molecules, e.g., drug molecules that a user of the system desires to manufacture. For example, the one or more labels can be labels such that, if associated with a biological sequence, indicate that molecules including the biological sequence, or otherwise defined by the biological sequence, are likely to be industrial enzymes, e.g., industrial enzymes that a user of the system desires to manufacture.

The system determines a predicted binding affinity of the molecule that includes the input biological sequence for each candidate target molecule in a collection of candidate target molecules (904). In some implementations, the molecule including the input biological sequence can be an agonist or antagonist of a receptor. In some implementations, the molecule including the input biological sequence can be an antibody or aptamer.

As an example, the system can determine the predicted binding affinity of the molecule that includes the input biological sequence for each candidate target molecule using a model that is configured to process an input including data representing a molecule and a target molecule to generate as output a predicted binding affinity of the molecule for the target molecule.

The system determines whether to select each candidate target molecule based at least in part on the predicted binding affinity of the molecule that includes the input biological sequence for each candidate target molecule (906). The system determines whether to select each candidate target molecule as a target molecule to which the molecule that includes the input biological sequence is predicted to bind.

For example, the system can determine, for each candidate target molecule, to select the candidate target molecule if the predicted binding affinity of the molecule including the input biological sequence for the candidate target molecule is above a binding affinity threshold. For example, the system can determine to select candidate target molecules that have the highest predicted binding affinities of the candidate target molecules in the collection of candidate target molecules (e.g., each selected candidate target molecule has a higher predicted binding affinity than each of the candidate target molecules in the collection which are not selected). The system can determine to select any suitable number of candidate target molecules.

The system identifies one or more candidate target molecules to which the molecule that includes the input biological sequence is predicted to bind (908). The system can identify the one or more candidate target molecules to be the candidate target molecules that the system has determined to select as target molecules to which the molecule that includes the input biological sequence are predicted to bind at the operation 906.

In some implementations, the molecule that includes the input biological sequence can be a polypeptide, a polynucleoside, or a polynucleotide, or an antibody, or an aptamer. In implementations in which the molecule that includes the input biological sequence is an agonist or antagonist of a receptor, the identified one or more target molecules can include the receptor. In implementations in which the molecule that includes the input biological sequence is an antibody or aptamer, the identified one or more target molecules can each include a respective antibody or aptamer target, in particular a virus or cancer cell protein, to which the molecule including the input biological sequence binds to provide a therapeutic effect.

In some implementations, the system physically synthesizes the molecule that includes the input biological sequence for use in treating one or more diseases associated with a target molecule selected from the identified one or more candidate target molecules (910).

In implementations in which the molecule that includes the input biological sequence is a drug molecule, the system performs physical experiments on physically synthesized instances of the drug molecule (912). The physical experiments are performed by the system to determine one or more of: absorption properties of the drug molecule, or distribution properties of the drug molecule, or metabolism properties of the drug molecule, or excretion properties of the drug molecule, or toxicity properties of the drug molecule.

In some implementations, the process 900 can include additional operations, fewer operations, or some of the operations can be divided into multiple operations. For instance, the process can include operations 902, 904, 906, and 908, optionally including operations 910 and 912.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework or a Jax framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more computers, the method comprising:
receiving a request to identify one or more labels associated with an input biological sequence; and
in response to receiving the request:
determining, for each of a plurality of candidate labels, a score characterizing a likelihood that the input biological sequence is associated with the candidate label, comprising, for each of the plurality of candidate labels:
identifying a plurality of positive biological sequences that are each associated with the candidate label; and
processing a network input comprising: (i) the input biological sequence, and (ii) the plurality of positive biological sequences, using a neural network and in accordance with values of a set of neural network parameters to generate the score characterizing the likelihood that the input biological sequence is associated with the candidate label; and
selecting one or more of the plurality of candidate labels as labels for the input biological sequence based on the scores.

2. The method of claim 1, further comprising, for each of the plurality of candidate labels, identifying a plurality of negative biological sequences that are each not associated with the candidate label;
wherein for each of the plurality of candidate labels, the network input to the neural network further comprises the plurality of negative biological sequences.

3. The method of claim 1, wherein for each of the plurality of candidate labels, the network input to the neural network includes labeling data that identifies each of the plurality of positive biological sequences as being associated with the candidate label.

4. The method of claim 1, wherein for each of the plurality of candidate labels, the network input to the neural network further comprises one or more of:
data characterizing a three-dimensional (3D) structure of a molecule includes the input biological sequence; or
for one or more of the plurality of positive biological sequences, data characterizing a respective 3D structure of a molecule that includes the positive biological sequence.

5. The method of claim 1, wherein for each of the plurality of candidate labels, identifying the plurality of positive biological sequences that are each associated with the candidate label comprises:
determining, for each candidate positive biological sequence in a set of candidate positive biological sequences that are associated with the candidate label, a respective similarity score that measures a similarity between: (i) the candidate positive biological sequence, and (ii) the input biological sequence; and
selecting the plurality of positive biological sequences as a proper subset of the set of candidate positive biological sequences based on the similarity scores.

6. The method of claim 5, wherein selecting the plurality of positive biological sequences as a proper subset of the set of candidate positive biological sequences based on the similarity scores comprises:
selecting a plurality of highest ranked candidate positive biological sequences under a ranking of the set of candidate positive biological sequences based on the similarity scores.

7. The method of claim 5, wherein selecting the plurality of positive biological sequences as a proper subset of the set of candidate positive biological sequences based on the similarity scores comprises:
stochastically sampling the plurality of positive biological sequences from the set of candidate positive biological sequences based on the similarity scores.

8. The method of claim 2, wherein for each of the plurality of candidate labels, identifying the plurality of negative biological sequences that are each not associated with the candidate label comprises:
determining, for each candidate negative biological sequence in a set of candidate negative biological sequences that are not associated with the candidate label, a respective similarity score that measures a similarity between: (i) the candidate negative biological sequence, and (ii) the input biological sequence; and
selecting the plurality of negative biological sequences as a proper subset of the set of candidate negative biological sequences based on the similarity scores.

9. The method of claim 8, wherein selecting the plurality of negative biological sequences as a proper subset of the set of candidate negative biological sequences based on the similarity scores comprises:
selecting a plurality of highest ranked candidate negative biological sequences under a ranking of the set of candidate negative biological sequences based on the similarity scores.

10. The method of claim 8, wherein selecting the plurality of negative biological sequences as a proper subset of the set of candidate negative biological sequences based on the similarity scores comprises:
stochastically sampling the plurality of negative biological sequences from the set of candidate negative biological sequences based on the similarity scores.

11. The method of claim 1, wherein the neural network has been trained by operations comprising:
pre-training the neural network to perform a pre-training task comprising processing a network input that includes a pair of training biological sequences to generate a predicted similarity score that is a prediction for a similarity between the pair of input biological sequences; and
fine-tuning the neural network to perform a fine-tuning task of predicting labels for training biological sequences.

12. The method of claim 11, wherein pre-training the neural network to perform the pre-training task further comprises:
partially masking one or both training biological sequences of the pair of training biological sequences prior to providing the pair of training biological sequences as a network input to the neural network; and
generating, using the neural network, a prediction for an unmasked version of any masked portions of the pair of training biological sequences.

13. The method of claim 1, further comprising, in response to receiving the request:
selecting the plurality of candidate labels for the input biological sequence as a proper subset of a set of possible labels for the input biological sequence, comprising:
determining, for each example biological sequence in a set of example biological sequences, a respective similarity score that measures a similarity between: (i) the example biological sequence, and (ii) the input biological sequence;
selecting a proper subset of the set of example biological sequences based on the similarity scores; and
identifying each label that is associated with at least one of the example biological sequences in the selected proper subset of the set of example biological sequences as a candidate label for the input biological sequence.

14. The method of claim 13, wherein selecting the proper subset of the set of example biological sequences based on the scores comprises:
selecting a plurality of highest ranked example biological sequences under a ranking of the set of example biological sequences based on the similarity scores.

15. The method of claim 1, further comprising:
selecting the input biological sequence as a drug target or substrate of an industrial enzyme based at least in part on the one or more labels selected for the input biological sequence; and
identifying one or more ligands that are predicted to bind to a molecule that includes the input biological sequence; and
physically synthesizing each of the one or more ligands that are predicted to bind to the molecule that includes the input biological sequence.

16. The method of claim 15, wherein identifying one or more ligands that are predicted to bind to a molecule that includes the input biological sequence comprises, for each candidate ligand in a collection of candidate ligands:
determining a predicted binding affinity of the ligand for the molecule that includes the input biological sequence; and
determining whether to select the candidate ligand as a ligand that is predicted to bind to the molecule that includes the input biological sequence based at least in part on the predicted binding affinity.

17. The method of claim 1, further comprising:
selecting the input biological sequence for inclusion in a molecule, or selecting a molecule that is otherwise defined by the input sequence, based at least in part on the one or more labels selected for the input biological sequence; and
identifying one or more candidate target molecules to which a molecule that includes the input biological sequence is predicted to bind, the molecule that includes the input biological sequence being a drug molecule or an industrial enzyme and each candidate target molecule being a candidate target molecule of the drug molecule or a candidate substrate molecule of the industrial enzyme; and
physically synthesizing the molecule that includes the input biological sequence for use in treating one or more diseases associated with a target molecule selected from the identified one or more candidate target molecules.

18. The method of claim 17, wherein identifying one or more candidate target molecules to which the molecule that includes the input biological sequence is predicted to bind comprises, for each candidate target molecule in a collection of candidate target molecules:
determining a predicted binding affinity of the molecule that includes the input biological sequence for the candidate target molecule; and
determining whether to select the candidate target molecule to which the molecule that includes the input biological sequence is predicted to bind based at least in part on the predicted binding affinity.

19. A system comprising one or more computers and one or more storage devices communicatively coupled to the one or more computers, wherein the one or more storage devices store instructions that, when executed by the one or more computers, cause the one or more computers to perform operations comprising:
receiving a request to identify one or more labels associated with an input biological sequence; and
in response to receiving the request:
determining, for each of a plurality of candidate labels, a score characterizing a likelihood that the input biological sequence is associated with the candidate label, comprising, for each of the plurality of candidate labels:
identifying a plurality of positive biological sequences that are each associated with the candidate label; and
processing a network input comprising: (i) the input biological sequence, and (ii) the plurality of positive biological sequences, using a neural network and in accordance with values of a set of neural network parameters to generate the score characterizing the likelihood that the input biological sequence is associated with the candidate label; and
selecting one or more of the plurality of candidate labels as labels for the input biological sequence based on the scores.

20. One or more non-transitory computer storage media storing instructions that when executed by one or more computers cause the one or more computers to perform operations comprising:
receiving a request to identify one or more labels associated with an input biological sequence; and
in response to receiving the request:
determining, for each of a plurality of candidate labels, a score characterizing a likelihood that the input biological sequence is associated with the candidate label, comprising, for each of the plurality of candidate labels:
identifying a plurality of positive biological sequences that are each associated with the candidate label; and
processing a network input comprising: (i) the input biological sequence, and (ii) the plurality of positive biological sequences, using a neural network and in accordance with values of a set of neural network parameters to generate the score characterizing the likelihood that the input biological sequence is associated with the candidate label; and
selecting one or more of the plurality of candidate labels as labels for the input biological sequence based on the scores.
